SEQUENCE LISTING (22 sequences)

SEQ ID NO: 1 - DNA, 81 bp, Homo sapiens
  source 1..81: mol_type = other DNA
ggcagcgtaggcgggagtggacttagcccctgtggttaatattcaccagcagcctcccccgttgcccctcactgacctggg

SEQ ID NO: 2 - DNA, 186 bp, Homo sapiens
  source 1..186: mol_type = other DNA
gggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 3 - DNA, 152 bp, Homo sapiens
  source 1..152: mol_type = other DNA
gggcgactcagatcccagccagtggacttagcccctgtttgctcctccgataactggggtgaccttggttaatattcaccagcagcctcccccgttgcccctctggatccactgcttaaatacggacgaggacagggccctgtctcctcagc

SEQ ID NO: 4 - DNA, 13 bp, Homo sapiens
  source 1..13: mol_type = other DNA
tggactttgcact

SEQ ID NO: 5 - skipped

SEQ ID NO: 6 - DNA, 10 bp, Homo sapiens
  source 1..10: mol_type = other DNA
attacgtaac

SEQ ID NO: 7 - DNA, 10 bp, Homo sapiens
  source 1..10: mol_type = other DNA
attgcacaat

SEQ ID NO: 8 - DNA, 13 bp, Homo sapiens
  source 1..13: mol_type = other DNA
gttaatgattaac

SEQ ID NO: 9 - DNA, 54 bp, Synthetic construct
  source 1..54: mol_type = other DNA
tggactttgcactattgcacaattgtaaacagttaatcattaacattacgtaac

SEQ ID NO: 10 - DNA, 139 bp, Synthetic construct
  source 1..139: mol_type = other DNA
tggactttgcactattgcacaattgtaaacagttaatcattaacattacgtaacttaaggcagcgtaggcgggagtggacttagcccctgtggttaatattcaccagcagcctcccccgttgcccctcactgacctggg

SEQ ID NO: 11 - DNA, 244 bp, Synthetic construct
  source 1..244: mol_type = other DNA
tggactttgcactattgcacaattgtaaacagttaatcattaacattacgtaacttaagggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 12 - DNA, 210 bp, Synthetic construct
  source 1..210: mol_type = other DNA
tggactttgcactattgcacaattgtaaacagttaatcattaacattacgtaacttaagggcgactcagatcccagccagtggacttagcccctgtttgctcctccgataactggggtgaccttggttaatattcaccagcagcctcccccgttgcccctctggatccactgcttaaatacggacgaggacagggccctgtctcctcagc

SEQ ID NO: 13 - DNA, 29 bp, Synthetic construct
  source 1..29: mol_type = other DNA
acccttaagcccagccagtggacttagcc

SEQ ID NO: 14 - DNA, 29 bp, Synthetic construct
  source 1..29: mol_type = other DNA
accactagtgattcactgtcccaggtcag

SEQ ID NO: 15 - DNA, 28 bp, Synthetic construct
  source 1..28: mol_type = other DNA
acccttaaggcagcgtaggcgggagtgg

SEQ ID NO: 16 - DNA, 30 bp, Synthetic construct
  source 1..30: mol_type = other DNA
accactagtcccaggtcagtgaggggcaac

SEQ ID NO: 17 - DNA, 28 bp, Synthetic construct
  source 1..28: mol_type = other DNA
acccttaagggcgactcagatcccagcc

SEQ ID NO: 18 - DNA, 31 bp, Synthetic construct
  source 1..31: mol_type = other DNA
accactagtgctgaggagacagggccctgtc

SEQ ID NO: 19 - DNA, 58 bp, Synthetic construct
  source 1..58: mol_type = other DNA
agcttggactttgcactattgcacaattgtaaacagttaatcattaacattacgtaac

SEQ ID NO: 20 - DNA, 58 bp, Synthetic construct
  source 1..58: mol_type = other DNA
ttaagttacgtaatgttaatgattaactgtttacaattgtgcaatagtgcaaagtcca

SEQ ID NO: 21 - DNA, 20 bp, Synthetic construct
  source 1..20: mol_type = other DNA
ccagcagctacttcaccaac

SEQ ID NO: 22 - DNA, 20 bp, Synthetic construct
  source 1..20: mol_type = other DNA
atgctagtcagcaggctctt

(19) United States
(12) Patent Application Publication
(10) Pub. No.: US 2026/0022398 A1
(43) Pub. Date: Jan. 22, 2026

(54) ENGINEERED LIVER-SPECIFIC CORE PROMOTERS AND THEIR APPLICATIONS

(71) Applicant: Sichuan Real & Best Biotech Co., Ltd., Chengdu (CN)

(72) Inventors: Biao Dong, Chengdu (CN); Li Yang, Chengdu (CN)

(73) Assignee: Sichuan Real&Best Biotech Co., Ltd., Chengdu (CN)

(21) Appl. No.: 19/107,527

(22) PCT Filed: May 10, 2024
(86) PCT No.: PCT/CN2024/092204

§ 371 Date: Feb. 27, 2025

Related U.S. Application Data
(60) Provisional application No. 63/510,974, filed on Jun. 29, 2023.

(30) Foreign Application Priority Data
May 11, 2023 (WO) PCT/CN2023/093412

(51) Int. Cl. C12N 15/86; A61K 38/37; A61K 48/00

(52) U.S. Cl. CPC C12N 15/86; A61K 38/37; A61K 48/0058; C12N 2750/14143; C12N 2830/008; C12N 2830/15

(57) ABSTRACT

The present invention relates to engineered liver-specific core promoters, synthetic promoters (which contains the engineered core promoters and enhancers), expression vectors (which contains the synthetic promoter), as well as methods of using the promoter or the expression vector thereof to address the need in the field, including treatment of various genetic diseases or conditions associated with the liver. In some embodiments, the liver-specific promoter includes continuous or discontinuous genome sequences from SERPINA1 genome.

### CROSS-REFERENCE TO RELATED APPLICATIONS

The present application claims the benefit of PCT Application No. PCT/CN2023/093412, filed on May 11, 2023, and U.S. Application No. 63/510,974 filed on Jun. 29, 2023, the contents of which are hereby incorporated by reference in their entirety.

### FIELD OF THE INVENTION

The invention relates generally to biotechnology, especially to gene expression regulation, gene therapy, and medicine. More particularly, disclosed herein are engineered liver-specific core promoters and their applications.

### BACKGROUND OF THE INVENTION

Recombinant AAV (rAAV) vectors are typically produced by replacing the viral coding sequences of the adeno-associated virus (AAV) with transgenes of interest. rAAV vectors are believed to be the most promising viral vector to treat genetic diseases. However, rAAV has a limited cargo capacity of less than 5.0 kb. If the size of the transgenes of interest is larger than 5.0 kb, rAAV vectors cannot be fully packaged in the AAV capsids, thus cannot be used for efficient gene therapy.

rAAV vector is usually composed of an expression cassette for the therapeutic gene and two ITRs. The expression cassette includes a promoter, a gene of interest (transgene), and a polyA sequence. The shortest polyA sequence available now is around 50 base pairs (bp). The two ITRs are located at both ends of the expression cassette. Each ITR is 145 bp long, and they are the packaging signals that must be incorporated in the rAAV vector. Thus, the space allocated for a therapeutic gene expression cassette is around 4.71 kb in length.

Some DNA fragments encoding therapeutic proteins are oversized for the rAAV packaging capacity. To keep the expression cassette in the range of efficient packaging, the size of promoter is important. A short promoter is preferable. In addition, to ensure safety and therapeutic efficiency, a promoter should be liver-specific and drive robust gene expression, since administration of a large amount of rAAV vectors may elicit adverse immune responses. As such, in the context of rAAV gene therapy for hemophilia A (HA), short, liver-specific and robust promoters are urgently needed.

In addition, although some DNA fragments encoding therapeutic proteins are small enough for efficient packaging, the encoded proteins' activities remain low. Consequently, a large amount of rAAV vectors must be injected into the patients to produce sufficient therapeutic proteins. However, administration of a large amount of rAAV vectors may elicit adverse immune responses. Thus, there exists an urgent need to develop robust, tissue-specific promoters and enhancers to enhance the activity of the encoded therapeutic proteins.

### SUMMARY OF THE INVENTION

The present invention provides engineered liver-specific core promoters, synthetic promoters, which contains the engineered core promoters and enhancers, expression vectors containing the synthetic promoters, as well as methods of using the promoter or the expression vector to address the need in the field, such as treatment of various genetic diseases or conditions.

In one aspect, the present invention provides an engineered core promoter.

In some embodiments, the core promoter is a liver-specific promoter.

In some embodiments, the engineered core promoter comprises a nucleic acid sequence of SEQ ID NO: 1 or SEQ ID NO: 3, or a nucleic acid sequence having at least 70%, 75%, 80%, 85%, 90%, 95%, 99%, or 100% sequence identity to the sequence as set forth in SEQ ID NO: 1 or 3.

The engineered core promoter comprising the sequence of SEQ ID NO: 1 is an 81 bp hAATsh core promoter, which contains four parts of the complementary genome sequences of Chr14: 94388570-94388757 (NC_000014.9, 94388743-94388757, 94388709-94388725, 94388644-94388680, 94388570-94388581). As shown in FIG. 1, the 4 parts are 15 bp, 17 bp, 37 bp, and 12 bp long, respectively. The engineered core promoter comprising the sequence of SEQ ID NO: 2 is a 186 bp hAATI core promoter, which contains the complementary genome sequences of Chr14: 94388560-94388745 (NC_000014.9). The engineered core promoter comprising the sequence of SEQ ID NO: 3 is a 152 bp hAATs2 core promoter, which contains the complementary genome sequences of Chr14: 94388594-94388745. See FIG. 1.

In another aspect, the present invention provides a synthetic promoter comprising the engineered core promoter disclosed herein and a nucleic acid sequence of an enhancer.

In some embodiments, the enhancer is an engineered enhancer comprising one or more DNA binding sites for transcription factors. Examples of the transcription factor include, but not limited to, HNF-4α, HNF-3β, D site-binding protein (DBP), CCAAT enhancer binding protein alpha/beta (C/EBP-α/β), and hepatocyte nuclearfactor 1 alpha/beta (HNF-1 α/β). The sequences of TFBS of HNF-4α, HNF-3β, DBP, C/EBP-α/β and HNF-1α/β selected in this invention are shown in SEQ ID NOs: 4-8, respectively.

In some embodiments, DNA binding sites for the above-mentioned transcription factors (TFBS) were combined, and subsequently modified. The ATG triple nucleotides were removed by rearranging the TFBS of the above-mentioned transcription factors and replacing the seventh base guanine in the sequences of TFBS of HNF-1α/β with cytosine, thereby, forming a 54 bp engineered enhancer (Em, SEQ ID NO: 9). Such enhancer was then added upstream of a core promoter to enhance transgene overexpression.

In some embodiments, the engineered enhancer comprises a nucleic acid sequence of SEQ ID NO: 9, or a nucleic acid sequence having at least 80%, 85%, 90%, 95%, 99%, or 100% sequence identity to the sequence of SEQ ID NO: 9.

In some embodiments, the synthetic promoter comprises a nucleic acid sequence of SEQ ID NO: 10 or SEQ ID NO: 12, or a nucleic acid sequence having at least 70%, 75%, 80%, 85%, 90%, 95%, 99%, or 100% sequence identity to the sequence as set forth in SEQ ID NO: 10 or SEQ ID NO: 12.

In another aspect, the present invention provides an expression vector comprising the synthetic promoter disclosed herein operably linked to a gene of interest (transgene).

In some embodiments, the expression vector is a plasmid, a recombinant retroviral vector, a recombinant lentiviral vector, a recombinant adenoviral vector, or a recombinant adeno-associated viral vector (rAAV). In a preferred embodiment, the expression vector is an rAAV.

In another aspect, the present invention provides a method of treating a genetic disease or condition in a subject in need thereof, comprising administering an expression vector disclosed herein to the subject, thereby expressing a therapeutic transgene in the subject's liver.

In some embodiments, the subject is a mammal. Preferably, the mammal is a human.

Examples of the genetic disease or condition associated with the liver include, but not limited to, genetic cholestasis, hemophilia A, hemophilia B, phenylketonuria, hereditary hemochromatosis, tyrosinemia type 1, alpha-1 antitrypsin deficiency, argininosuccinic aciduria, liver cancer, glycogen storage disease, urea cycle disorder, Crigler-Najjar syndrome, familial amyloid polyneuropathy, atypical hemolytic uremic syndrome-1, primary hyperoxaluria type 1, maple syrup urine disease, acute intermittent porphyria, coagulation defects, GSD type1A, homozygous familial hypercholesterolemia, organic acidurias, cystic fibrosis, erythropoietic protoporphyria, Gaucher disease, familial hypercholesterolemia, ornithine and transcarbamylase deficiency.

In another aspect, the present invention provides use of the promoter, or the expression vectors disclosed herein in enhancing the expression level of a transgene in a liver cell, wherein the nucleic acid comprises a transgene operably linked to the promoter.

In another aspect, the present invention provides use of the promoter, or the expression vector, or the pharmaceutical composition provided herein for the manufacture of a medicament for the genetic disease or condition associated with the liver.

In another aspect, the present invention provides a kit comprising the promoter, or the expression vector, or the pharmaceutical composition disclosed herein.

In some embodiments, the kit further comprises instructions for using the kit.

The subsequent description and appended claims will provide a better understanding of the features, aspects, and advantages of the present invention.

### BRIEF DESCRIPTION OF DRAWINGS

The present invention will now be described by way of non-limitative example with reference to the following figures, in which:

FIG. 1 is a diagram of regulatory elements for driving transgene overexpression in rAAV vector. Enh, Enhancer; Pro, Promoter; Int, Intron; pA, polyA; HCR, complementary sequence of partial coding sequence (CDS) of hepatic control region HCR-1; Ex, proximal element X located in 5′ untranslated region (5′ UTR) of human SERPINA1 (encoding human α-1 antitrypsin) genome; HEx, 66 bp combined enhancer containing HCR and Ex; HLP, 252 bp, a positive control synthetic promoter; hAATs2, 152 bp hAAT (human α-1 antitrypsin) core promoter; hAATI, 186 bp core promoter consistent with the core promoter of HLP synthetic promoter; hAATsh, 81 bp short core promoter containing four parts of human SERPINA1 genome sequences; FRE72, 119 bp, a short promoter containing two parts of human SERPINA1 genome sequences.

FIG. 2 is a diagram of synthetic promoters for driving transgene overexpression in rAAV vector. HCR, complementary sequence of partial coding sequence (CDS) of hepatic control region HCR-1; Ex, proximal element X located in 5′ untranslated region (5′ UTR) of human SERPINA1 (encoding human α-1 antitrypsin) genome; HLP, 252 bp, a positive control synthetic promoter; Em-hAATs2, 210 bp synthetic promoter containing 152 bp human α-1 antitrypsin short core promoter and 54 bp enhancer Em; Em-hAATI, 244 bp synthetic promoter containing 186 bp human α-1 antitrypsin short core promoter and 54 bp enhancer Em; Em-hAATsh, 139 bp synthetic promoter containing 81 bp human α-1 antitrypsin short core promoter and 54 bp enhancer Em.

FIG. 3 shows the activities of liver-specific core promoters in cells. Different rAAV vector plasmids were transfected in Huh7 cells on 12-well plates. The cells of negative control group transfected with pssAAV-CB-EGFP. Cells were collected 24 hours post-transfection to measure the activities of luciferase. hAATsh, 81 bp short core promoter derived from SERPINA1 (encoding human α-1 antitrypsin) genome; hAATI, 186 bp core promoter consistent with the core promoter of HLP chimeric promoter; hAATs2, 152 bp core promoter. n=4, *P<0.05, **P<0.01 and ns represent not significant between FRE72 and other promoter groups (or between other two promoter groups) using a two-tailed Student's t-test.

FIG. 4 shows the activities of synthetic promoters in cells. Different rAAV vector plasmids were transfected in Huh7 cells on 12-well plates. The cells of the negative control group were transfected with pssAAV-CB-EGFP. Cells were collected 24 hours post-transfection to measure the activities of luciferase. HLP, 252 bp, a positive control synthetic promoter; Em-hAATsh, 139 bp synthetic promoter containing 81 bp human α-1 antitrypsin short promoter and 54 bp enhancer Em; Em-hAATI, 244 bp synthetic promoter containing 186 bp human α-1 antitrypsin short promoter and 54 bp enhancer Em; Em-hAATs2, 210 bp synthetic promoter containing 152 bp human α-1 antitrypsin short core promoter and enhancer Em. n=4, ***P<0.001, ****P<0.0001 and ns represent not significant between HLP and other promoter groups using a two-tailed Student's t-test.

FIGS. 5A and 5B show the activities of synthetic promoters in factor VIII deficient mice. Around 6-8 weeks of factor VIII deficient mice were treated with different rAAVs via tail vein injection. Plasma of mice were collected for measuring the activities and protein levels of F VIII-SQ by APTT and ELISA. Em, the 54 bp enhancer; Em-hAATsh, 139 bp synthetic promoter containing 81 bp human α-1 antitrypsin short promoter and 54 bp enhancer Em; Em-hAATI, 244 bp synthetic promoter containing 186 bp human α-1 antitrypsin short promoter and 54 bp enhancer Em; Em-hAATs2, 210 bp synthetic promoter containing 152 bp human α-1 antitrypsin short core promoter and enhancer Em. n=3, *P<0.05 and ns represent not significant between two promoter groups using a two-tailed Student's t-test.

### DETAILED DESCRIPTION OF THE INVENTION

References to specific features of the invention are made in the summary section above, and the detailed description section and claims below. It is to be understood that the disclosure of the invention in this specification includes all possible combinations of such specific features. For example, where a specific feature is disclosed in the context of a particular aspect or embodiment of the invention, or a particular claim, the feature can also be used, to the extent possible, in combination with and/or in the context of other aspects and embodiments of the invention, and in the invention generally.

Gene therapy is a promising method to treat genetic diseases. In gene therapy, a gene of interest (transgene) is introduced into one or more recipient cells, and expression of the introduced gene in the recipient cell affects the cell's function and results in a therapeutic effect in a subject. rAAV vectors are believed to be the most promising viral vector to be used in gene therapy. However, rAAV has a limited cargo capacity of less than 5.0 kb.

Except for other necessary elements, the space for the length of a promoter and a therapeutic gene is around 4.71 kb. Thus, the shorter the promoter, the more space for the therapeutic gene. Also, a liver-specific promoter is preferable as it prevents gene expression in organs other than liver, thereby, ensuring the safety of the therapy. In addition, a promoter would be more effective if it drives robust gene expression, thereby, ensuring the therapeutic efficiency.

In one aspect, the present invention provides an engineered core promoter comprising the 81 bp hAATsh core promoter (SEQ ID NO: 1) or the 152 bp hAATs2 core promoter (SEQ ID NO: 3). The 81 bp hAATsh core promoter contains four parts of the complementary genome sequences of Chr14: 94388570-94388757 (NC_000014.9, 94388743-94388757, 94388709-94388725, 94388644-94388680, 94388570-94388581), upstream sequences of human SERPINA1 genome (encoding human α-1 antitrypsin, hAAT). The 152 bp hAATs2 core promoter (SEQ ID NO: 3) contains the complementary genome sequences of Chr14: 94388594-94388745.

As used herein, an engineered core promoter (also known as the minimal-region) usually contains a TATA-box necessary for recruiting RNA polymerase II and the assembly of general transcription factors to form the preinitiation complex. An “engineered core promoter” is sometimes called an “artificial core promoter” or “synthetic core promoter.”

In some embodiments, the core promoter is a liver-specific promoter.

In some embodiments, the core promoter comprises a nucleic acid sequence of SEQ ID NO: 1 or SEQ ID NO: 3, or a nucleic acid sequence having at least 80%, 85%, 90%, 95%, 99%, or 100% sequence identity to the sequence of SEQ ID NO: 1 or SEQ ID NO: 3. The hAAT1 core promoter comprising the nucleic acid sequence of SEQ ID NO: 2 has been used as a control in this invention.

SEQ ID NO: 1:
hAATsh core promoter (81 bp)
GGCAGCGTAGGCGGGAGTGGACTTAGCCCCTGTGGTTAATAT
TCACCAGCAGCCTCCCCCGTTGCCCCTCACTGACCTGGG
SEQ ID NO: 2:
hAAT1 core promoter (186 bp)
GGGCGACTCAGATCCCAGCCAGTGGACTTAGCCCCTGTTTGC
TCCTCCGATAACTGGGGTGACCTTGGTTAATATTCACCAGCA
GCCTCCCCCGTTGCCCCTCTGGATCCACTGCTTAAATACGGA
CGAGGACAGGGCCCTGTCTCCTCAGCTTCAGGCACCACCACT
GACCTGGGACAGTGAATC
SEQ ID NO: 3:
hAATs2 core promoter (152 bp)
GGGCGACTCAGATCCCAGCCAGTGGACTTAGCCCCTGTTTGC
TCCTCCGATAACTGGGGTGACCTTGGTTAATATTCACCAGCA
GCCTCCCCCGTTGCCCCTCTGGATCCACTGCTTAAATACGGA
CGAGGACAGGGCCCTGTCTCCTCAGC

In another aspect, the present invention provides a synthetic promoter comprising the engineered core promoter disclosed herein and a nucleic acid sequence of an enhancer.

As used herein, a “synthetic promoter” is a stretch of DNA comprising a core promoter and a combination of heterologous upstream regulatory elements (cis-motifs or transcription factor-binding sites). A “synthetic promoter” is sometimes called an “chimeric promoter”, “engineered promoter” or “artificial promoter.” The core promoter usually contains a TATA-box necessary for recruiting RNA polymerase II and the assembly of general transcription factors to form the preinitiation complex. A synthetic promoter may comprise, for example, regions of known promoters, regulatory elements, transcription factor binding sites, enhancer elements, repressor elements, and the like.

In some embodiments, the core promoter is a liver-specific promoter.

A liver-specific promoter primarily directs transgene expression in the liver, but it may also lead to transgene expression in other tissues or organs at lower levels. Use of a liver-specific promoter in the expression cassette can restrict unwanted transgene expression in other tissues and facilitate persistent transgene expression in liver.

As used herein, an “enhancer” is a nucleic acid sequence that increases the rate of transcription by increasing the activity of a promoter. An “engineered enhancer” refers to a man-made enhancer that is not occurring naturally. An “engineered enhancer” is sometimes called an “artificial enhancer” or “synthetic enhancer.”

Transcription factors are proteins that bind to specific DNA sequences and thereby control the transfer (or transcription) of genetic information from DNA to RNA. The “DNA binding sites” used herein refer to the specific DNA sequences that transcription factors bind to.

In some embodiments, the transcription factor is selected from the group consisting of hepatocyte nuclear factor-4 α (HNF-4α), hepatocyte nuclear factor 3-beta (HNF-3β), D site-binding protein (DBP), CCAAT enhancer binding protein alpha (C/EBP-α), CCAAT enhancer binding protein beta (C/EBP-β), hepatocyte nuclear factor 1 alpha (HNF-1α), and hepatocyte nuclear factor 1 beta (HNF-1β).

HNF-4α is encoded by the HNF4A gene and is a nuclear transcription factor that binds DNA as a homodimer. It controls the expression of several genes, including hepatocyte nuclear factor 1 alpha, a transcription factor which regulates the expression of several hepatic genes.

HNF-3β is encoded by the FOXA2 gene and is a member of the forkhead class of DNA-binding proteins. These hepatocyte nuclear factors are transcriptional activators for liver-specific genes such as albumin and transthyretin.

DBP is a transcriptional activator that recognizes and binds to the sequence 5′-RTTAYGTAAY-3′ found in the promoter of genes such as albumin, CYP2A4 and CYP2A5.

C/EBP-α contains a basic leucine zipper (bZIP) domain and recognizes the CCAAT motif in the promoters of target genes. It functions in homodimers and heterodimers with CCAAT/enhancer-binding proteins β and γ. Activity of this protein can modulate the expression of genes involved in cell cycle regulation as well as in body weight homeostasis.

C/EBP-β is a bZIP transcription factor that can bind as a homodimer to certain DNA regulatory regions. It can also form heterodimers with the related proteins C/EBP-α, C/EBP-δ, and C/EBP-γ.

HNF-1α is encoded by the HNF1A gene and is a transcription factor that is highly expressed in the liver and is involved in the regulation of the expression of several liver-specific genes.

HNF-1β is encoded by the HNF1B gene and is a protein of the homeobox-containing basic helix-turn-helix family. The HNF1B protein is believed to form heterodimers with another member of this transcription factor family, HNF-1α.

In some embodiments, the DNA binding sites for HNF-4α, HNF-3β, DBP, C/EBP-α/β and HNF-1α/β comprise sequences of SEQ ID NO: 4, SEQ ID NO: 5, SEQ ID NO: 6, SEQ ID NO: 7 and SEQ ID NO: 8, respectively.

SEQ ID NO: 4:
HNF-4 α transcription
factor DNA binding site (TFBS)
TGGACTTTGCACT
SEQ ID NO: 5:
HNF-3 β TFBS
TGTAAACA
SEQ ID NO: 6:
DBP TFβS
ATTACGTAAC
SEQ ID NO: 7:
C/EBP- α/β TFBS
ATTGCACAAT
SEQ ID NO: 8:
HNF-1 α/β TFBS
GTTAATGATTAAC

In some embodiments, the engineered enhancer comprises a sequence of SEQ ID NO: 9, or a nucleic acid sequence having at least 70%, 75%, 80%, 85%, 90%, 95%, 99%, or 100% sequence identity to the sequence of SEQ ID NO: 9.

SEQ ID NO: 9:
Em enhancer
TGGACTTTGCACTATTGCACAATTGTAAACAGTTAATCATTAACA
TTACGTAAC

The Em enhancer is 54 bp and contains, from 5′ end to 3′ end, the DNA binding sites of HNF-4α, C/EBP-α/β, HNF-3β, HNF-1α/β (the seventh base guanine was converted to cytosine), and DBP.

In some embodiments, the synthetic promoter comprises a nucleic acid sequence of SEQ ID NO: 10 or SEQ ID NO: 12, or a nucleic acid sequence having at least 70%, 75%, 80%, 85%, 90%, 95%, 99%, or 100% sequence identity to the sequence of SEQ ID NO: 10 or 12. The Em-hAAT1 synthetic promoter comprising the nucleic acid sequence of SEQ ID NO: 11 has been used as a control in this invention.

SEQ ID NO: 10:
Em-hAATsh (139 bp, synthetic promoter)
TGGACTTTGCACTATTGCACAATTGTAAACAGTTAATCATTAACA
TTACGTAACttaaGGCAGCGTAGGCGGGAGTGGACTTAGCCCCTG
TGGTTAATATTCACCAGCAGCCTCCCCCGTTGCCCCTCACTGACC
TGGG

The Em-hAATsh synthetic promoter is 139 bp long and includes the 54 bp enhancer Em and the 81 bp core promoter hAATsh, connected by necessary enzyme digestion sites.

SEQ ID NO: 11:
Em-hAAT1 (244 bp, synthetic promoter)
TGGACTTTGCACTATTGCACAATTGTAAACAGTTAATCATTAACA
TTACGTAACttaaGGGCGACTCAGATCCCAGCCAGTGGACTTAGC
CCCTGTTTGCTCCTCCGATAACTGGGGTGACCTTGGTTAATATTC
ACCAGCAGCCTCCCCCGTTGCCCCTCTGGATCCACTGCTTAAATA
CGGACGAGGACAGGGCCCTGTCTCCTCAGCTTCAGGCACCACCAC
TGACCTGGGACAGTGAATC

The Em-hAAT1 synthetic promoter is 244 bp long and includes the 54 bp engineered enhancer Em, and the 186 bp core promoter hAAT1, connected by necessary enzyme digestion sites.

SEQ ID NO: 12:
Em-hAATs2 (210 bp, synthetic promoter)
TGGACTTTGCACTATTGCACAATTGTAAACAGTTAATCATTAACA
TTACGTAACTTAAGGGCGACTCAGATCCCAGCCAGTGGACTTAGC
CCCTGTTTGCTCCTCCGATAACTGGGGTGACCTTGGTTAATATTC
ACCAGCAGCCTCCCCCGTTGCCCCTCTGGATCCACTGCTTAAATA
CGGACGAGGACAGGGCCCTGTCTCCTCAGC

The Em-hAATs2 synthetic promoter is 210 bp long and includes the 54 bp enhancer Em and the 152 bp promoter hAATs2, connected by necessary enzyme digestion sites.

In another aspect, the present invention provides an expression vector comprising the synthetic promoter disclosed herein operably linked to a gene of interest.

The term “operably linked” means that the regulatory sequences necessary for expression of a coding sequence are placed in the DNA molecule in the appropriate positions relative to the coding sequence so as to effect expression of the coding sequence.

In some embodiments, the expression vector is a plasmid, a recombinant retroviral vector, a recombinant lentiviral vector, a recombinant adenoviral vector, or a recombinant adeno-associated viral vector (rAAV).

Human adeno-associated virus (AAV) is a non-pathogenic parvovirus that only productively replicates in cells co-infected by a helper virus, usually adenovirus or herpes virus. The virus has a wide host range and can productively infect many cell types from a variety of animal species. Nevertheless, AAV has not been implicated in any human or animal disease.

AAV binds to cells via a heparan sulfate proteoglycan receptor. Once attached, AAV entry is dependent upon the presence of a co-receptor, either the fibroblast growth factor receptor or αvβ5 integrin molecule. In infected cells, the incoming AAV single-stranded DNA (ssDNA) is converted to double-stranded transcriptional template. Cells infected with AAV and a helper virus will undergo productive replication of AAV prior to cell lysis, which is induced by the helper virus rather than AAV. Helper virus encodes proteins or RNA transcripts which are transcriptional regulators and are involved in DNA replication or modify the cellular environment in order to permit efficient viral production.

Recombinant AAV (rAAV) vectors are typically produced by replacing the viral coding sequences with transgenes of interest. These vectors have been shown to be highly efficient for gene transfer and expression at a number of different sites in vitro and in vivo. They have consistently mediated stable expression and have been safe in studies performed in the respiratory tract, the central nervous system, skeletal muscle, liver, and eye. The efficiency of rAAV-mediated transduction has increased as the titer and purity of rAAV preparations has improved.

The inverted terminal repeats (ITRs) from the AAV genome are the only viral sequences required in cis to generate rAAV vectors. Recombinant constructs containing two ITRs bracketing a gene expression cassette of ˜5 kb are converted into a ssDNA vector genome and packaged into AAV particles in the presence of AAV rep and cap gene products and helper functions. Methods or production and purification of rAAV are known in the art.

One of the genes of interest can be the F8 gene, which is located on the X chromosome and encodes Factor VIII (FVIII). FVIII is one of the main factors for coagulation cascade. The loss-of-function mutation of FVIII causes a genetic disease called Hemophilia A (HA). The occurrence rate for HA is 1/5000 in males.

The common treatment for HA is replacement therapy. Concentrates of factor VIII are slowly dripped or injected into a vein of HA patients. These infusions help replace the factor VIII that is missing or low in a patient. However, this replacement therapy may generate inhibitors of the injected or acquired factor VIII, leading to the failure of this replacement therapy.

An alternative therapy for hemophilia A is gene therapy based on rAAV vectors. The rAAV vectors allow long-term, stable expression of transgenes in vivo for therapeutic purposes. The coding region of FVIII is 7035 bp long and can be divided into 6 domains, namely, A1, A2, B, A3, C1, C2. For rAAV vectors to be efficiently packaged into adeno-associated virus (AAV) capsids, the size of the expression cassette containing a therapeutic gene should generally not exceed 5 kb.

Due to the limitation of AAV packaging capacity, the full-length FVIII coding region will not be effectively packaged into AAV vectors. To overcome this problem, researchers must reduce the size of the coding region of FVIII. Previous studies have shown that B domain of FVIII (908 aa) can be replaced with SQdomain (14 aa), which does not seem to be required for FVIII clotting activity. This engineered FVIII is known as FVIII-SQ and has 6 domains, i.e., A1, A2, SQ, A3, C1, and C2 domains. A1, A2, and SQdomains form the heavy chain and A3, C1, C2 form the light chain of FVIII-SQ. The nucleotide encoding the FVIII-SQ is 4374 bp, which allow it to be efficiently inserted into rAAV vectors.

However, even when the expression cassette is around 5 kb, many packaged rAAV vectors remain incomplete and defective, and these defective rAAV vectors cannot produce functional FVIII. To address this limitation, a large amount of rAAV vectors need to be injected to HA patients to produce sufficient functional FVIII. However, administration of a large amount of rAAV vectors may elicit adverse immune responses.

To address these limitations, it is essential to maintain the size of both the promoter and the polyA tail as minimal as possible. In some embodiments, the synthetic promoters disclosed herein are used to direct the expression of FVIII-SQ or other engineered FVIII fragments. Due to the reduced size of these synthetic promoters, the expression cassette exhibits higher packaging rate.

In another aspect, the present invention provides pharmaceutical compositions for delivering a transgene described herein to a subject, including a human subject. In some embodiments, the composition comprises any of the nucleic acids or vectors described herein. In some embodiments, pharmaceutical compositions disclosed herein comprise any vector disclosed herein and one or more pharmaceutically acceptable carrier. In some embodiments, the composition comprises any of the AAV vectors described herein. In some embodiments, pharmaceutical compositions disclosed herein comprise any AAV vectors disclosed herein and one or more pharmaceutically acceptable carrier.

Although the descriptions of pharmaceutical compositions provided herein, e.g., AAV vectors, are principally directed to pharmaceutical compositions that are suitable for administration to humans, a skilled artisan in the field would understand that such compositions are generally suitable for administration to any other animals, e.g., to non-human animals, or non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are to be administered to humans.

In another aspect, the present invention provides a method of treating a genetic disease or condition in a subject in need thereof, comprising administering an expression vector disclosed herein to the subject, thereby expressing a therapeutic protein in the subject's liver.

In some embodiments, the subject is a mammal. In some embodiments, the subject is a human.

Examples of the genetic disease or condition associated with the liver include, but are not limited to, genetic cholestasis, hemophilia A, hemophilia B, phenylketonuria, hereditary hemochromatosis, tyrosinemia type 1, alpha-1 antitrypsin deficiency, argininosuccinic aciduria, liver cancer, glycogen storage disease, urea cycle disorder, Crigler-Najjar syndrome, familial amyloid polyneuropathy, atypical hemolytic uremic syndrome-1, primary hyperoxaluria type 1, maple syrup urine disease, acute intermittent porphyria, coagulation defects, GSD type1A, homozygous familial hypercholesterolemia, organic acidurias, cystic fibrosis, erythropoietic protoporphyria, Gaucher disease, familial hypercholesterolemia, ornithine and transcarbamylase deficiency.

In some embodiments, methods provided herein can be used to treat a genetic disease or condition in a subject in need, comprising administering an expression vector disclosed herein to the subject, thereby expressing a therapeutic protein in the subject's liver.

In another aspect, the present invention provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the pharmaceutical compositions or vectors of the present disclosure may be included in a kit. In some embodiments, kits can further include reagents and/or instructions for creating and/or synthesizing compounds and/or pharmaceutical compositions of the present disclosure. In some embodiments, kits can also include one or more buffers. In some embodiments, kits of the disclosure can include components for making protein or nucleic acid arrays or libraries and thus, may include, for example, solid supports.

In some embodiments, kit components can be packaged either in aqueous media or in lyophilized form. The container means of the kits will generally include at least one vial, test tube, flask, bottle, syringe or other container means, into which a component can be placed, and suitably aliquoted. Where there is more than one kit component, (labeling reagent and label may be packaged together), kits can also generally contain second, third or other additional containers into which additional components may be separately placed. In some embodiments, kits can also include a second container means for containing sterile, pharmaceutically acceptable buffers and/or other diluents. In some embodiments, various combinations of components can be included in one or more vials. Kits of the present disclosure can also typically include means for containing compounds and/or pharmaceutical compositions of the present disclosure, e.g., proteins, nucleic acids, and any other reagent containers in close confinement for commercial sale. Such containers can include injection or blow-molded plastic containers into which desired vials are retained.

In some embodiments, kit components are provided in one and/or more liquid solutions. In some embodiments, liquid solutions are aqueous solutions, with sterile aqueous solutions being particularly used. In some embodiments, kit components can be provided as dried powder(s). When reagents and/or components are provided as dry powders, such powders can be reconstituted by the addition of suitable volumes of solvent. In some embodiments, it is envisioned that solvents can also be provided in another container means.

In some embodiments, kits can include instructions for employing kit components as well the use of any other reagent not included in the kit. Instructions can include variations that may be implemented.

### Definition

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein, the terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

5′ and/or 3′: Nucleic acid molecules (such as, DNA and RNA) are said to have “5′ ends” and “3′ ends” because mononucleotides are reacted to make polynucleotides in a manner such that the 5′ phosphate of one mononucleotide pentose ring is attached to the 3′ oxygen of its neighbor in one direction via a phosphodiester linkage. Therefore, one end of a linear polynucleotide is referred to as the “5′ end” when its 5′ phosphate is not linked to the 3′ oxygen of a mononucleotide pentose ring. The other end of a polynucleotide is referred to as the “3′ end” when its 3′ oxygen is not linked to a 5′ phosphate of another mononucleotide pentose ring. Notwithstanding that a 5′ phosphate of one mononucleotide pentose ring is attached to the 3′ oxygen of its neighbor, an internal nucleic acid sequence also may be said to have 5′ and 3′ ends.

In either a linear or circular nucleic acid molecule, discrete internal elements are referred to as being “upstream” or 5′ of the “downstream” or 3′ elements. With regard to DNA, this terminology reflects that transcription proceeds in a 5′ to 3′ direction along a DNA strand. Promoter and enhancer elements, which direct transcription of a linked gene, are generally located 5′ or upstream of the coding region. However, enhancer elements can exert their effect even when located 3′ of the promoter element and the coding region. Transcription termination and polyadenylation signals are located 3′ or downstream of the coding region.

The term “promoter region” or “promoter” refers to a region of DNA that directs/initiates transcription of a nucleic acid (e.g., a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription. A tissue-specific promoter is a promoter that directs/initiated transcription primarily in a single type of tissue or cell. For example, a liver-specific promoter is a promoter that directs/initiates transcription in liver tissue to a substantially greater extent than other tissue types.

The term “enhancer” refers to a nucleic acid sequence that increases the rate of transcription by increasing the activity of a promoter.

As is well known in the art, most eukaryotic genes contain both exons and introns. The term “exon” refers to a nucleic acid sequence found in genomic DNA that is bioinformatically predicted and/or experimentally confirmed to contribute a contiguous sequence to a mature mRNA transcript. The term “intron” refers to a nucleic acid sequence found in genomic DNA that is predicted and/or confirmed not to contribute to a mature mRNA transcript, but rather to be “spliced out” during processing of the transcript.

The term “vector” refers to a small carrier DNA molecule into which a DNA sequence can be inserted for introduction into a host cell where it will be replicated. An “expression vector” is a specialized vector that contains a gene or nucleic acid sequence with the necessary regulatory regions needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g., promoters, enhancers, and termination elements) in an expression vector. This definition is also sometimes applied to the arrangement of nucleic acid sequences of a first and a second nucleic acid molecule wherein a hybrid nucleic acid molecule is generated.

As used herein, the term “percent identical” is used herein with reference to comparisons among nucleic acid or amino acid sequences. It is defined as the percentage of nucleotides or amino acid residues in a candidate sequence that are identical with the nucleotides or amino acid residues in a specific sequence, after aligning the sequences and introducing gaps, if necessary, to achieve the maximum percent sequence identity. Nucleic acid and amino acid sequences are often compared using computer programs that align sequences of nucleic or amino acids thus defining the differences between the two. Comparisons of nucleic acid or amino acid sequences may be performed in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

The phrase “sequence identity” refers to the identity or similarity between two or more nucleic acid sequences, or two or more amino acid sequences, which is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g., deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS] dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddlTP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (RGG), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl) aminonaphthalene-1-sulfonic acid (EDANS). Specific examples of fluorescently labeled nucleotides can include [R6G] dUTP, [TAMRA] dUTP, [R110] dCTP, [R6G] dCTP, [TAMRA] dCTP, [JOE] ddATP, [R6G] ddATP, [FAM] ddCTP, [R110] ddCTP, [TAMRA] ddGTP, [ROX] ddTTP, [dR6G] ddATP, [dR110] ddCTP, [dTAMRA] ddGTP, and [dROX] ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Illinois; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-LJTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

The term “cDNA (complementary DNA)” refers to a piece of DNA lacking internal, non-coding segments (introns) and regulatory sequences that determine transcription. cDNA is synthesized in the laboratory by reverse transcription from messenger RNA extracted from cells. cDNA can also contain untranslated regions (UTRs) that are responsible for translational control in the corresponding RNA molecule.

The term “gene,” as used herein, refers to a nucleic acid (e.g., DNA such as genomic DNA and cDNA) and its corresponding nucleotide sequence that is involved in encoding an RNA transcript. The term used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

“Transcription factor (TF)” is a protein that binds to specific DNA sequences and thereby controls the transfer (or transcription) of genetic information from DNA to RNA. TFs perform this function alone or with other proteins in a complex, by promoting (as an activator), or blocking (as a repressor) the recruitment of RNA polymerase (the enzyme that performs the transcription of genetic information from DNA to RNA) to specific genes. The specific DNA sequences to which a TF binds is known as a response element (RE) or regulatory element. Other names include cis-element and cis-acting transcriptional regulatory element.

Gene therapy involves the introduction of a heterologous nucleic acid molecule into one or more recipient cells, wherein expression of the heterologous nucleic acid in the recipient cell affects the cell's function and results in a therapeutic effect in a subject. For example, the heterologous nucleic acid molecule may encode a protein, which affects the function of the recipient cell.

“Inverted terminal repeat (ITR)” refers to symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

The term “control” as used herein refers to a reference standard.

Hemophilia is a blood coagulation disorder caused by a deficient clotting factor activity, which decreases hemostasis. Severe forms result when the concentration of clotting factor is less than about 1% of the normal concentration of the clotting factor in a normal subject. In some subjects, hemophilia is due to a genetic mutation which results in impaired expression of a clotting factor. In others, hemophilia is an auto-immune disorder, referred to as acquired hemophilia, in which the antibodies which are generated against a clotting factor in a subject result in decreased hemostasis.

Hemophilia A results from a deficiency of functional clotting Factor VIII, while hemophilia B results from a deficiency of functional clotting Factor IX. These conditions which are due to a genetic mutation are caused by an inherited sex-linked recessive trait with the defective gene located on the X chromosome, and this disease is therefore generally found only in males. The severity of symptoms can vary with this disease, and the severe forms become apparent early on. Bleeding is the hallmark of the disease and typically occurs when a male infant is circumcised. Additional bleeding manifestations make their appearance when the infant becomes mobile. Mild cases may go unnoticed until later in life when they occur in response to surgery or trauma. Internal bleeding may happen anywhere, and bleeding into joints is common.

“Factor VIII deficiency,” as used herein, includes deficiency in clotting activity caused by production of defective factor VIII, by inadequate or no production of factor VIII, or by partial or total inhibition of factor VIII by inhibitors. Hemophilia A is a type of factor VIII deficiency resulting from a defect in an X-linked gene and the absence or deficiency of the factor VIII protein it encodes.

The terms “derivative,” “variant” and “fragment” when used herein with reference to a polypeptide, refers to a polypeptide related to a wild type of polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a referenced polypeptide.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “pharmaceutical composition” refers to a mixture of the expression vectors disclosed herein or the rAAV vectors disclosed herein with other chemical components, such as diluents or carriers. The pharmaceutical composition facilitates administration of the compound to an organism. Pharmaceutical compositions will generally be tailored to the specific intended route of administration. A pharmaceutical composition is suitable for human and/or veterinary applications.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The terms “treatment” and “treating,” as used herein, refer to an approach for obtaining beneficial or desired results including, but not limited to, a therapeutic benefit and/or a prophylactic benefit. For example, a treatment can comprise administering a system or cell population disclosed herein. A therapeutic benefit can refer to any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the quantity of a composition, for example a composition comprising rAAV vectors, that is sufficient to result in a desired activity upon administration to a subject in need thereof. The term “therapeutically effective” can refer to a quantity of a composition that is sufficient to delay the manifestation, arrest the progression, relieve or alleviate at least one symptom of a disorder treated by the methods of the present disclosure.

A “therapeutic effect” may occur if there is a change in the condition being treated. The change may be positive or negative. For example, a “positive effect” may correspond to an increase in the number of activated T-cells in a subject. In another example, a ‘negative effect’ may correspond to a decrease in the amount or size of a tumor in a subject. A “change” in the condition being treated, may refer to at least 1%, 2%, 3%, 4%, 5%, 6%, 7%, 8%, 9%, 10%, 25%, 50%, 75%, or 100% change in the condition. The change can be based on improvements in the severity of the treated condition in an individual, or on a difference in the frequency of improved conditions in populations of individuals with and without the administration of a therapy. Similarly, a method of the present disclosure may comprise administering to a subject a number of cells that are “therapeutically effective.” The term “therapeutically effective” should be understood to have a definition corresponding to having a therapeutic effect.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

### Example 1. Designing and Obtaining Liver Specific Core Promoters and Testing their Effects

This example describes how the liver-specific core promoters were designed and obtained, and their promoting activities were tested. Specifically, synthetic promoters, each comprised of a short core promoter and an engineered enhancer, were utilized to drive expression of a B-domain deleted FVIII (FVIII-SQ) in mice, or luciferase overexpression in Huh7 cells. The activities and protein levels of FVIII-SQ and luciferase activities were measured to compare the activities of synthetic promoters that have different combination of core promoters and enhancers. Results show that the synthetic promoters had significantly higher promoting activities than the 119 bp FRE72 liver-specific promoter and the 252 bp HLP liver-specific promoter.

### Methods

### Liver-Specific Core Promoter Design

Conservation of sequences between Homo sapiens and Mus musculus, and the position of TATA box related to the transcription start site (TSS) were taken into consideration of choosing promoters in this invention.

The 81 bp hAATsh core promoter (SEQ ID NO: 1) contains four parts of the complementary genome sequences of Chr14: 94388570-94388757 (NC_000014.9, 94388743-94388757, 94388709-94388725, 94388644-94388680, 94388570-94388581), upstream sequences of human SERPINA1 genome (encoding human α-1 antitrypsin, hAAT). The 186 bp hAATI core promoter (SEQ ID NO: 2) contains the complementary genome sequences of Chr14: 94388560-94388745 (NC_000014.9) and the 152 bp hAATs2 core promoter (SEQ ID NO: 3) contains the complementary genome sequences of Chr14: 94388594-94388745.

The short and liver-specific FRE72 promoter was chosen as a positive control core promoter in accordance with the patent application WO 2021/084277 A2. The liver-specific HLP promoter was chosen as a positive control synthetic promoter according to the reference paper (Blood. 2013 Apr. 25; 121(17): 3335-3344.).

### Specific Enhancer Design

Several transcription factors including hepatocyte nuclear factor 1 alpha/beta (HNF-1α/β), HNF-3β, HNF-4α, CCAAT enhancer binding protein alpha/beta (C/EBP-α/β), and D site-binding protein (DBP) were selected and used in this invention.

In this invention, DNA binding sites of the above-mentioned transcription factors (TFBS) were arranged in combination to form a 54 bp engineered enhancer (Em, SEQ ID NO: 9), which was then added upstream of a core promoter to enhance transgene overexpression. The sequences of TFBS of HNF-4α, HNF-3β, DBP, C/EBP-α/β and HNF-1α/β selected in this invention are shown in SEQ ID NOs: 4-8, respectively. The Em contains, from 5′ end to 3′ end, the DNA binding sites of HNF-4α, C/EBP-α/β, HNF-3β, HNF-1α/β (the seventh base guanine was converted to cytosine), and DBP.

Construction of rAAV Vector Plasmids

The HLP, FRE72, hAATsh promoters were synthesized and cloned into pUC-HLP plasmid by GENERAL BIOSYSTEMS (Anhui, China).

The vector pssAAV-HLP—FVIII-SQwas digested by Mlu I and Nhe I double enzymes, then HLP fragment was cloned into the pssAAV-MSP-luciferase-A vector backbone followed by HindiIII and Spe I digestions to obtain pssAAV-HLP-luciferase-A. pssAAV-MSP-luciferase-A is a plasmid expression vector containing a MSP promoter, an intron, luciferase coding sequences and a BGH polyA tail and ampicillin was used for selection.

The HLP fragment obtained from pssAAV-HLP-luciferase-A plasmid was cloned into pssAAV-MSP-luciferase-K vector backbone by Pacl and Stul double enzymes digestion to obtain pssAAV-HLP-luciferase-K. pssAAV-MSP-luciferase-K is a plasmid expression vector containing an MSP promoter, an intron, luciferase coding sequences and a BGH polyA tail and kanamycin was used for selection.

The fragment of FRE72 promoter was amplified by polymerase chain reaction (PCR) using the pUC-FRE72 plasmid as the template. The fragment of hAATsh promoter was amplified by PCR using the pUC-hAATsh plasmid as the template. Fragments of hAATI and hAATs2 promoterswere obtained by PCR using the pUC-HLP plasmid as the template. pssAAV-HLP2-luciferase-K plasmid vector backbone digested by Afl II and Spe I enzymes was inserted the digested fragments of FRE72, hAATsh, hAATI or hAATs2 promoters to obtain pssAAV-FRE72-luciferase-K, pssAAV-hAATsh-luciferase-K, pssAAV-hAATI-luciferase-K, pssAAV-hAATs2-luciferase-K. The sequences of the three core promoters, hAATsh, hAATI and hAATs2, are shown in SEQ ID NO: 1-3.

The three plasmid vectors including pssAAV-hAATsh-luciferase-K, pssAAV-hAATI-luciferase-K, and pssAAV-hAATs2-luciferase-K were digested by Hind III and Af II double enzymes and used for insertion of the enhancer Em to construct pssAAV-Em-hAATsh-luciferase-K, pssAAV-Em-hAATI-luciferase-K and pssAAV-Em-hAATs2-luciferase-K. The sequences of three synthetic promoters, Em-hAATsh, Em-hAATI and Em-hAATs2, are shown in SEQ ID NO: 10-12.

Three fragments (Em-hAATsh, Em-hAATI and Em-hAATsh) digested by Padl and Spel double restriction enzymes digestion were cloned into pssAAV-Es-hAATs2-FVIII-SQ vector backbone followed by Padl and Nhel enzymes digestion to obtain pssAAV-Em-hAATsh-FVIII-SQ pssAAV-Em-hAATI—FVIII-SQ and pssAAV-Em-hAATs2-FVII-SQ recombinant vectors.

All primers used for vector plasmids construction were listed in Table 1 (SEQ ID NOs: 13-20).

TABLE 1
Primers for construction of the core
and synthetic promoters

| SEQ | | |
|---|---|---|
| ID | | Primer sequence |
| NO: | Primer name | (5′-3′) |
| 13 | AflII-FRE72-F | ACCCTTAAGCCCAGCCAGTG |
| | | GACTTAGCC |
| 14 | SpeI-FRE72-I-R | ACCACTAGTGATTCACTGTC |
| | | CCAGGTCAG |
| 15 | AflII-hAATsh-F | ACCCTTAAGGCAGCGTAGGC |
| | | GGGAGTGG |
| 16 | SpeI-hAATsh-R | ACCACTAGTCCCAGGTCAGT |
| | | GAGGGGCAAC |
| 17 | AflII-hAATI-s2-F | ACCCTTAAGGGCGACTCAGA |
| | | TCCCAGCC |
| 18 | SpeI-hAATs2-R | ACCACTAGTGCTGAGGAGAC |
| | | AGGGCCCTGTC |
| 19 | Em-HindII-F | AGCTTGGACTTTGCACTATT |
| | | GCACAATTGTAAACAGTTA |
| | | ATCATTAACATTACGTAAC |
| 20 | Em-AflII-R | TTAAGTTACGTAATGTTAA |
| | | TGATTAACTGTTTACAATT |
| | | GTGCAATAGTGCAAAGTCC |
| | | A |
| 21 | FVIII-SQ-qPCR-F | CCAGCAGCTACTTCACCAAC |
| 22 | FVIII-SQ-qPCR-R | ATGCTAGTCAGCAGGCTCTT |

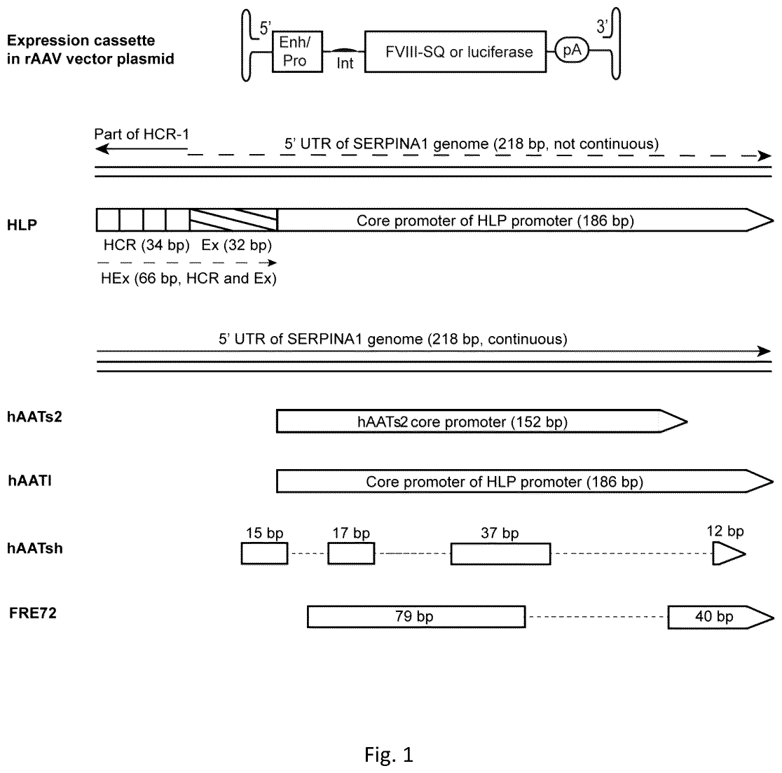

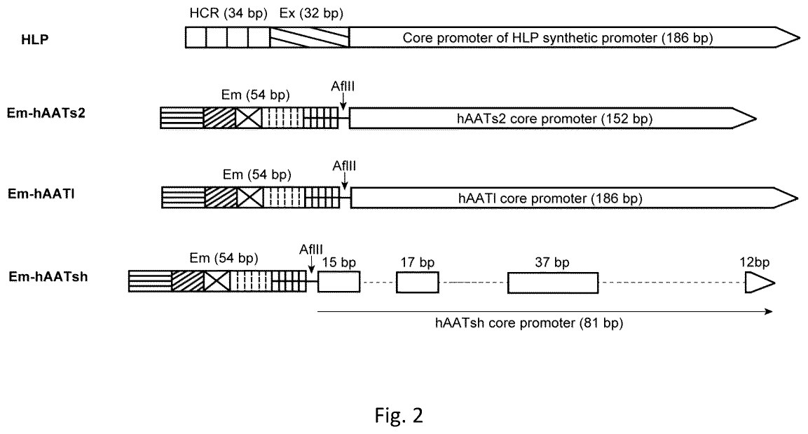

FIG. 1 shows the diagrams of the core promoters and FIG. 2 shows the diagrams of synthetic promoters for driving luciferase or FVIII-SQ expression in rAAV vector.

### Cell Culture and Transfection

Huh7 cells were obtained from ATCC and cultured in DMEM (Dulbecco's Modified Eagle Medium) containing 10% FBS (Fetal Bovine Serum) and 1% Penicillin-Streptomycin. Cells were incubated at 37° C. with 5% COz.

Transfection was performed on 12-well plates. Briefly, the Huh7 cells were grown overnight until the confluency was around 80%, then the mixture of 0.5 μg plasmid expressing luciferase or EGFP (Enhanced Green Fluorescent Protein) and 1.5 μL of PElpro (Polyplus transfection, Illkirch) was added to each well according to the manufacturer's protocol. The cells were transfected with each plasmid in quadruplicate. pssAAV-CB-EGFP plasmid was transfected to serve as a negative control of luciferase expression. After 6-8 hours, medium containing transfection reagents and plasmids were removed and fresh DMEM containing FBS and Penicillin-Streptomycin were added on 12-well plates. The cells were collected 24 h post transfection for measurement of the luciferase activity.

### Luciferase Assays

The transfected cells on 12-well plates were rinsed gently using DPBS after removal of DMEM, then DPBS was removed and 100 μL cell lysis buffer in Firefly Luciferase Reporter Gene Assay Kit (Beyotime, Shanghai) was added to each well. Samples were transferred into Eppendorf tubes after 5 min at room temperature. After centrifuging at 4° C. and 12,000 rpm for 2 min, the supernatants were transferred into new tubes. 30 μL of each sample was added 30 μL of substrate in Firefly Luciferase Receptor Gene Assay Kit on 96-well white assay plate (Corning, New York), and the mixture was used for detecting luciferase activities by a Synergy H1 Hybrid Multi-Mode Microplate Reader (BioTek, Winooski).

Package and Purification of rAAV Vectors

Three rAAV vectors, ssAAV8-Em-hAATsh-FVIII-SQ (Em-hAATsh), ssAAV8-Em-hAATI-FVIII-SQ (Em-hAATI), ssAAV8-Em-hAATs2-FVIII-SQ (Em-hAATs2), were packaged in HEK293 cells using the triple plasmids transfection method and were purified by two rounds cesium chloride ultracentrifugation. The titers of rAAV vectors were determined by qPCR using the forward primer FVIII-SQ-qPCR-F (SEQ ID NO: 21), and reverse primer FVIII-SQ-qPCR-R (SEQ ID NO: 22).

### Animals

Around 6-8 weeks of factor VIII deficient mice were injected with different rAAV vectors or PBS buffer via tail vein injection. Three mice were treated for each group. The injection dosage was 4×10¹¹ genome copies for each mouse. Plasma of mice were collected from posterior orbital venous plexus of mice before injection and 4 weeks post injection by adding the blood samples to 1.5 mL tubes prefilled anticoagulant sodium citrate (final concentration 3.8%). Then supernatants, plasma of mice, were transferred into new tubes following centrifugation for 15 min at 2500 g. Plasma samples diluted in appropriate proportions were utilized for measurement of the activities and protein levels of FVIII-SQ by APTT and ELISA.

### APTT (One Stage Activated Partial Thromboplastin Clotting Time)

ReFacto (Genetics Institute, Cambridge, MA) was serially diluted with factor VIII deficient plasma from 1 U/mL (200 ng/mL) by ½ till 1/64 dilutions and used as the standards. ReFacto is a recombinant FVIII and can be used as the standard for APTT and ELISA. 50 μL of STA-PTT reagent (Diagnostica Stago, Asnieres, France) was added to enough strips of STAGO cuvettes containing a magnetic bead in each well. Thereafter, each diluted standard protein and each diluted plasma sample was added to different wells of STAGO cuvettes all pre-filled with STA-PTT reagent. The mixtures were incubated at 37° C. for 170 seconds. Coagulation time was then initiated and measured by adding 50 μL of 25 mM CaCl₂ using STAGO machine (Diagnostica Stago, Asnieres, France). The FVIII-SQactivities were calculated according to the standard curve.

### ELISA (Enzyme-Linked Immunosorbent Assay)

In a 96-well plate, the wells were coated with 100 μL of the 2.5 ng/μL capture antibody PAH—FVIII-S(Haematologic Technologies, Essex) in coating buffer (containing 0.1 M sodium bicarbonate and carbonate, pH 9.6) for each well at 4° C. overnight. After the plates were washed 3 times for 5 minutes every time with 300 μL of PBST buffer (140 mM NaCl, 2.5 mM KCl, 8 mM Na₂HPO₄, 2 mM KH₂PO₄ and 0.05% Tween-20, pH 8.4), the wells were blocked with 300 μL of PBST buffer containing 3% BSA at room temperature for 2 hours. The wells are washed with PBST buffer three times, then 100 μL of the standards or samples were added and incubated at room temperature for 1.5 hours. Serial dilutions of ReFacto (12.5 ng/mL two-fold serially diluted to 0.1953 ng/mL) were used as the standards. After the wells were washed three times with PBST buffer, 100 μL of the 0.5 ng/μL detecting antibody biotin-labeled GMA-8021 (Green Mountain Antibodies, Burlington) was added. The plate was incubated at room temperature for 1 hour. After 3 times of washing, each well was added 100 μL 200-fold diluted Streptavidin-HRP (CST, Boston) in PBST buffer containing 0.1% BSA and incubated for 1 hour in the dark. Then the plate was washed three times with PBST buffer and the color was developed using 100 μL of KPL SureBlue TMB 1-Component Microwell Peroxidase Substrate (Seracare, Milford). The color development was carried out at room temperature for 1-10 min in the dark and stopped by adding 100 μL of 0.5 M H₂SO₄. The OD values were determined by a spectrophotometer at 450 nm and 630 nm. FVIII-SQ amounts in the medium were calculated according to the standard curve.

### Data Analysis

The schematic pictures were produced by Adobe Illustrator 2021. Data statistical analysis was calculated by GraphPad Prism 9.0.0. All data was reported as mean±SD. The significant differences in luciferase activities between FRE72 promoter or HLP promoter and other promoter groups (or between other two promoter groups) were calculated by a two-tailed Student's t-test. The significant differences in luciferase activities between two promoter groups were also calculated by a two-tailed Student's t-test.

### Results

### Activities of Engineered Liver-Specific Core Promoters

The cargo capacity of rAAV vectors is limited, and an expression cassette containing a regular size promoter and the therapeutic FVIII-SQ for treating Hemophilia A (HA) is too large to fit in. Thus, smaller size promoters that could drive FVIII-SQ overexpression effectively are in urgent need. A short enough promoter FRE72 (WO 2021/084277 A2, 119 bp) with compact regulatory elements was invented by other researchers and the FRE72 promoter was used as a positive control core promoter in present invention. Here, a shorter, liver-specific core promoter was obtained and some other promoters utilized for comparison were constructed (FIG. 1).

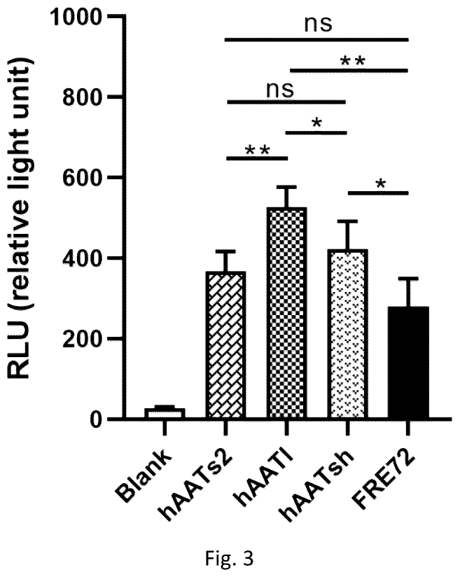

As shown in FIG. 3, luciferase activities driving by three engineered core promoters (hAATsh, hAATI and hAATs2) and the positive control core promoter FRE72 were both significantly increased compared with the negative control group in Huh7 cells. Interestingly, the 81 bp short core promoter hAATsh (SEQ ID NO: 1) was shorter but stronger than 119 bp FRE72 promoter for luciferase expression in Huh7 cells (P=0.0249). In addition, hAATI core promoter (SEQ ID NO: 2) showed significantly enhanced activities than FRE72 core promoter (P=0.0011) for driving luciferase expression while the luciferase activities of hAATs2 core promoter (SEQ ID NO: 3) was slightly increased compared with the FRE72 short core promoter (P=0.083). Besides, the activity of hAATI core promoter was higher than the activity of hAATsh (P=0.0481) and hAATs2 core promoter (P=0.0038) while there was no significant difference in luciferase activities between hAATsh and hAATs2 core promoter groups (P=0.2308).

### Comparison of the Activities of Synthetic Promoters in Huh7 Cells

To further enhance the activity of 81 bp hAATsh promoter, Em enhancer was added to the 5′ end of the hAATsh promoter to get a 139 bp Em-hAATsh (SEQ ID NO: 10) synthetic promoter (FIG. 2). Similarly, 244 bp Em-hAATI (SEQ ID NO: 11) and 210 bp Em-hAATs2 (SEQ ID NO: 12) synthetic promoters were also constructed by adding Em enhancer to the 5′ end of the hAATI and hAATs2 promoters, respectively (FIG. 2). The synthetic liver-specific HLP promoter (252 bp) was selected as a positive control synthetic promoter based on a scientific article (Blood. 2013 Apr. 25; 121(17): 3335-3344.). Comparison of the activities was done among the promoters of Em-hAATsh, Em-hAATI, Em-hAATs2, and HLP for luciferase expression in Huh7 cells.

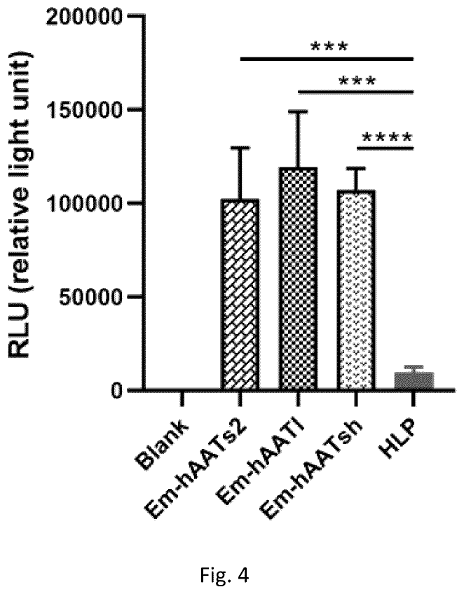

As seen in FIG. 4, under the control of three synthetic promoters (Em-hAATsh, Em-hAATI and Em-hAATs2), the luciferase activities were all around 10 times higher than those under the control of the HLP promoter (P<0.0001, P=0.0003, P=0.0005). In addition, the three synthetic promoters showed roughly equal activities for luciferase overexpression in Huh7 cells.

### Comparison of the Activities of Synthetic Promoters in Factor VIII Deficient Mice

Based on the positive results of the three synthetic promoters in Huh7 cells, it was hypothesized that they could all drive FVIII-SQ overexpression efficiently in factor VIII deficient mice. Thus, rAAV vectors including FVIII-SQ expression cassette driving by these three synthetic promoters (Em-hAATsh, Em-hAATI, and Em-hAATs2) were packaged and injected into factor VIII deficient mice. Plasma of mice before injection and 4 weeks post injection were both collected for comparison of the activities and protein levels of FVIII-SQ driven by different promoters.

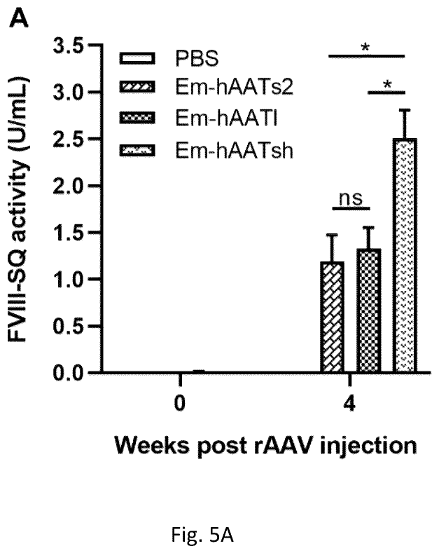

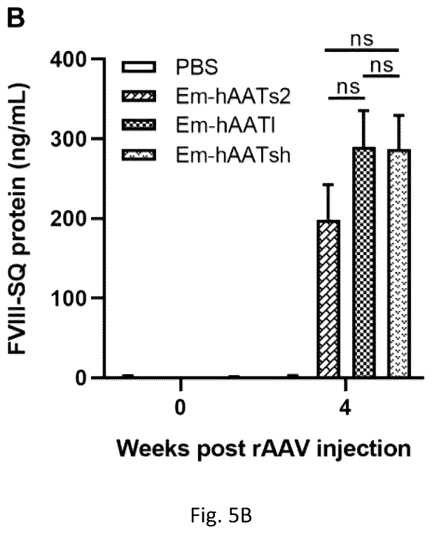

As shown in FIG. 5B, the protein levels of FVIII-SQ driven by Em-hAATsh (SEQ ID NO: 10) and Em-hAATI (SEQ ID NO: 11) synthetic promoters were both slightly higher than those driven by Em-hAATs2 (SEQ ID NO: 12) promoters but there were no significant differences among these three synthetic promoter groups. As shown in FIG. 5A, there was no significant difference in the activities of FVIII-SQ between Em-hAATI and Em-hAATs2 groups.

Interestingly, however, the activity of FVIII-SQ driven by Em-hAATsh promoter was significantly higher than the activities of FVIII-SQ driven by Em-hAATI (P=0.0235) and Em-hAATs2 promoters (P=0.0253) in mice, although there were no noteworthy differences among the activities of these same three synthetic promoters in Huh7 cells (compare FIG. 5A with FIG. 4). rAAV has a limited cargo capacity, and Em-hAATsh and Em-hAATs2 are both shorter than Em-hAATI synthetic promoters, especially Em-hAATsh is only 81 bp. One explanation for the different activities of these three synthetic promoters observed in mice is that more intact rAAV vectors containing the entire expression cassette of FVIII-SQ driving by Em-hAATsh and Em-hAATs2 might be packaged and delivered into mice than the rAAV vectors containing Em-hAATI synthetic promoters.

All in all, Em-hAATsh and Em-hAATs2, short and strong enough, were both suitable synthetic promoter for FVIII-SQ overexpression in rAAV vector. Em-hAATsh or Em-hAATs2 synthetic promoter was beneficial for efficient factor VIII therapy of Hemophilia A utilizing rAAV vector.

### Example 2. Treatment of a Patient Suffering from a Genetic Disease

A human patient is tested to have a genetic disease.

An expression vector is made comprising the synthetic promoter disclosed herein operably linked to a therapeutic transgene. The effectiveness and safety of the expression vector are tested in in vitro cell culture and in vivo animal models before being used to treat the human patient.

### Example 3. Treatment of Human Hemophilia a Patient Using an rAAV Expression Vector

This example describes an exemplary method for the clinical use of rAAV vectors encoding FVIII-SQfor the treatment of hemophilia A.

An expression vector is made comprising the synthetic promoter disclosed herein operably linked to FVIII-SQ. The effectiveness and safety of the expression vector are tested in in vitro cell culture and in vivo animal models before being used to treat the human patient.

A patient diagnosed with hemophilia A is selected for treatment. The patient is administered a therapeutically effective amount of rAAV. The rAAV can be administered intravenously. An appropriate therapeutic dose can be selected by a medical practitioner.

In some cases, the therapeutically effective dose is in the range of 1×10¹¹ to 1×10¹⁴ viral particles (vp)/kg, such as about 1×10¹¹ vp/kg. In most instances, the patient is administered a single dose. The health of the subject can be monitored over time to determine the effectiveness of the treatment.

1. An engineered core promoter comprising a nucleic acid sequence having at least 70%, 75%, 80%, 85%, 90%, 95%, 99%, or 100% sequence identity to a sequence as set forth in SEQ ID NO: 1 or SEQ ID NO: 3.

2. A synthetic promoter comprising the engineered core promoter of claim 1 and an enhancer.

3. The synthetic promoter of claim 2, wherein the enhancer is an engineered enhancer.

4. The synthetic promoter of claim 3, wherein the engineered enhancer comprises one or more DNA binding sites for transcription factors, wherein each transcription factor is selected from the group consisting of HNF-4α, HNF-3β, D site-binding protein (DBP), CCAAT enhancer binding protein alpha/beta (C/EBP-α/β), and hepatocyte nuclear factor 1 alpha/beta (HNF-1 α/β).

5. The synthetic promoter of claim 4, wherein the DNA binding sites for transcription factors HNF-4α, HNF-3β, DBP, C/EBP-α/β and HNF-1α/β comprise nucleic acid sequences of SEQ ID NO: 4, SEQ ID NO: 5, SEQ ID NO: 6, SEQ ID NO: 7, and SEQ ID NO: 8, respectively.

6. The synthetic promoter of claim 3, wherein the engineered enhancer comprises a nucleic acid sequence having at least 70%, 75%, 80%, 85%, 90%, 95%, 99%, or 100% sequence identity to a sequence as set forth in SEQ ID NO: 9.

7. The synthetic promoter of claim 2, wherein the synthetic promoter comprises a nucleic acid sequence having at least 70%, 75%, 80%, 85%, 90%, 95%, 99%, or 100% sequence identity to a sequence as set forth in SEQ ID NO: 10 or SEQ ID NO: 12.

8. An expression vector comprising an engineered core promoter of claim 1.

9. The expression vector of claim 8, further comprising a transgene operably linked to the engineered core promoter of claim 1.

10. The expression vector of claim 9, wherein the transgene encodes a therapeutic protein for treatment of a genetic disease or condition associated with liver.

11. The expression vector of claim 10, wherein the therapeutic protein is a Factor VIII protein or a functional fragment thereof.

12. The expression vector of claim 811, wherein the expression vector is a plasmid, a recombinant retroviral vector, a recombinant lentiviral vector, a recombinant adenoviral vector, or a recombinant adeno-associated viral vector (rAAV).

13. The expression vector of claim 12, wherein the expression vector is an rAAV vector.

14. A pharmaceutical composition comprising an engineered core promoter of claim 1, a synthetic promoter of claim 2, or an expression vector of claim 8, and a pharmaceutically acceptable carrier.

15. A method of treating a genetic disease or condition associated with liver, comprising administering a therapeutically effective amount of a pharmaceutical composition of claim 14 to a subject in need thereof.

16. The method of claim 15, wherein the subject is a mammal.

17. The method of claim 16, wherein the mammal is a human.

18. The method of claim 15, wherein the genetic disease or condition associated with liver is selected from the group consisting of genetic cholestasis, hemophilia A, hemophilia B, phenylketonuria, hereditary hemochromatosis, tyrosinemia type 1, alpha-1 antitrypsin deficiency, argininosuccinic aciduria, liver cancer, glycogen storage disease, urea cycle disorder, Crigler-Najjar syndrome, familial amyloid polyneuropathy, atypical hemolytic uremic syndrome-1, primary hyperoxaluria type 1, maple syrup urine disease, acute intermittent porphyria, coagulation defects, GSD type1A, homozygous familial hypercholesterolemia, organic acidurias, cystic fibrosis, erythropoietic protoporphyria, Gaucher disease, familial hypercholesterolemia, ornithine and transcarbamylase deficiency.

19. (canceled)

20. (canceled)

21. A kit comprising an engineered core promoter of claim 1 or a pharmaceutical composition of claim 14.

22. The kit of claim 21, further comprising instructions for using contents of the kit.
